(19) United States
(12) Patent Application Publication
(10) Pub. No.: US 2008/0125686 A1
(43) Pub. Date: May 29, 2008

(54) Heat mitigating hemostatic agent

(76) Inventor: Denny Lo, Bethlehem, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/606,617

(22) Filed: Nov. 29, 2006

(51) Int. Cl. A61F 13/00

(52) U.S. Cl. 602/48; 604/289

(57) ABSTRACT

A hemostatic agent in the form of particles comprises a first component and a second component bound thereto, each component having hemostatic properties. Additional components may also be included. The first component may be a zeolite and the second component may be clay. A device for promoting the clotting of blood comprises a receptacle for retaining particles of a hemostatic agent therein, at least a portion of the receptacle being defined by a mesh. A pad for controlling bleeding comprises a mesh structure defined by openings sized to accommodate the flow of blood therethrough and also by a hemostatic agent retained in the mesh structure. A bandage applicable to a bleeding wound comprises a substrate, a mesh mounted on the substrate, and a hemostatic agent retained in the mesh. The mesh is defined by a plurality of members arranged to define openings through which blood may flow.

### TECHNICAL FIELD

The present invention relates generally to devices for promoting hemostasis and, more particularly, to hemostatic agents in which exothermic reactions can be modulated and devices incorporating such agents.

### BACKGROUND OF THE INVENTION

Blood is a liquid tissue that includes red cells, white cells, corpuscles, and platelets dispersed in a liquid phase. The liquid phase is plasma, which includes acids, lipids, solubilzed electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, animals, including humans, can be wounded. Often bleeding is associated with such wounds. In some circumstances, the wound and the bleeding are minor, and normal blood clotting functions in addition to the application of simple first aid are all that is required. Unfortunately, however, in other circumstances substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stanch the flow of blood in a timely manner.

Moreover, severe wounds can often be inflicted in remote areas or in situations, such as on a battlefield, where adequate medical assistance is not immediately available. In these instances, it is important to stop bleeding, even in less severe wounds, long enough to allow the injured person or animal to receive medical attention.

In an effort to address the above-described problems, materials have been developed for controlling excessive bleeding in situations where conventional aid is unavailable or less than optimally effective. Although these materials have been shown to be somewhat successful, they are sometimes not effective enough for traumatic wounds and tend to be expensive. Furthermore, these materials are sometimes ineffective in some situations and can be difficult to apply as well as remove from a wound.

Additionally, or alternatively, the previously developed materials can produce undesirable side effects. For example, one type of prior art blood clotting material is generally a powder or a fine particulate in which the surface area of the material often produces an exothermic reaction upon the application of the material to blood. Oftentimes excess material is unnecessarily poured onto a wound, which can exacerbate the exothermic effects. Depending upon the specific attributes of the material, the resulting exothermia may be sufficient to cause discomfort to or even burn the patient. Although some prior art patents specifically recite the resulting exothermia as being a desirable feature that can provide clotting effects to the wound that are similar to cauterization, there exists the possibility that the tissue at and around the wound site may be undesirably impacted.

Some of the previously developed materials can also be difficult to apply and maintain in contact with the wound site. Also, to remove such materials from wounds, irrigation of the wound is often required. If an amount of material is administered that causes discomfort or burning, the wound may require immediate flushing. In instances where a wounded person or animal has not yet been transported to a facility capable of providing the needed irrigation, undesirable effects or over-treatment of the wound may result.

Bleeding can also be a problem during surgical procedures. Apart from suturing or stapling an incision or internally bleeding area, bleeding is often controlled using a sponge or other material used to exert pressure against the bleed site and/or absorb the blood. However, when the bleeding becomes excessive, these measures may not be sufficient to stop the flow of blood. Moreover, any highly exothermic bleed-control material may damage the tissue surrounding the bleed site and may not be configured for easy removal after use.

Based on the foregoing, it is a general object of the present invention to provide a hemostatic agent that overcomes or improves upon the drawbacks associated with the prior art. It is also a general object of the present invention to provide devices capable of applying such hemostatic agents.

### SUMMARY OF THE INVENTION

According to one aspect, the present invention resides in a hemostatic agent in the form of particles. Each particle comprises a first component and a second component bound thereto, each component having hemostatic properties. Additional components may also be included. Both (or all) components are uniformly and homogenously mixed in amounts to modulate an exothermic reaction when the hemostatic agent is applied to blood. When using the hemostatic agent to treat a bleeding wound, contacting the bleeding wound with said hemostatic agent causes blood flowing from said bleeding wound to clot.

According to another aspect, the present invention resides in a device for promoting the clotting of blood. The device comprises a receptacle for retaining a hemostatic agent in particle form therein, at least a portion of the receptacle being defined by a mesh having openings. The hemostatic agent comprises particles, each particle having a first component having hemostatic properties and a second component also having hemostatic properties, the second component binding the first component. The first component and the second component are uniformly and homogenously mixed in amounts to modulate an exothermic reaction when the hemostatic agent is applied to blood. When treating a bleeding wound, application of the device causes at least a portion of the hemostatic agent to come into contact with blood through the openings.

According to another aspect, the present invention resides in a pad for controlling bleeding. In such an aspect, the pad comprises a mesh structure defined by openings sized to accommodate the flow of blood therethrough and also by a hemostatic agent retained in the mesh structure. The hemostatic agent comprises particles, each particle having a first component having hemostatic properties and a second component also having hemostatic properties, the second component binding the first component. The first component and the second component are uniformly and homogenously mixed in amounts to modulate an exothermic reaction when the hemostatic agent is applied to blood. When treating a bleeding wound, application of the pad causes at least a portion of the hemostatic agent to come into contact with blood through the openings.

According to another aspect, the present invention resides in a bandage applicable to a bleeding wound. The bandage comprises a substrate, a mesh mounted on the substrate, and a hemostatic agent retained in the mesh. The mesh is defined by a plurality of members arranged to define openings, the openings being dimensioned to accommodate the flow of blood therethrough. The hemostatic agent comprises particles, each particle having a first component having hemostatic properties and a second component having hemostatic properties, the second component binding the first component. The first component and the second component are uniformly and homogenously mixed in amounts to modulate an exothermic reaction when the device is applied to a bleeding wound and the hemostatic agent comes into contact with blood.

In the preferred embodiments of the hemostatic agents and the devices disclosed herein, the first component may be, for example, a zeolite, and the second component may be, for example, a clay such as kaolin.

When the first and second components in the embodiments described herein are zeolite and clay, respectively, one advantage of the present invention is that the zeolite component in combination with the clay component causes less of an exothermic reaction with blood than if the zeolite was used alone. In particular, the presence of clay tempers the exothermic effects experienced at the wound site by causing a less aggressive drawing of moisture from the blood. It is theorized that the less aggressive drawing of moisture from the blood is the result of a less rapid transfer of moisture from the wound. However, the porous nature of the hemostatic agent still allows water to be wicked away to cause thickening of the blood, thereby facilitating the formation of clots.

Another advantage is that the hemostatic agent of the present invention reacts more exothermically with blood than does one that is all or substantially all clay material. A small amount of heat aids in the process of coagulating blood. Accordingly, by blending proportionate amounts of a component (e.g., zeolite) that produces an exothermic reaction with blood together with clay, the total amount of heat can be modulated and some amount of heat can be desirably generated to facilitate the clotting of the blood.

Another advantage is that the hemostatic properties of the hemostatic agent can be “tuned” depending on the needs at hand. This tuning can be easily effected by varying the ratio of the individual components in the agent. More particularly, the amount of zeolite relative to the clay can be adjusted to control the amount of heat generated at a wound site. Controlling the amount of heat at a wound site may be useful in the treatment of certain patients such as pediatric or geriatric patients or when the wound being treated is in a particularly sensitive or delicate area.

Still another advantage of the present invention is that the agents and devices of the present invention are easily applied to open wounds. Particularly when the hemostatic agent is retained in a mesh or similar device, the device can be readily removed from a sterilized packaging and placed or held directly at the points from which blood emanates to cause clotting.

### BRIEF DESCRIPTION OF THE DRAWINGS

FIG. 1 is a schematic representation of a blood clotting device of the present invention.

FIG. 2 is a side view of the blood clotting device of FIG. 1 illustrating the retaining of molecular sieve particles in a mesh container.

FIG. 3 is a side view of a pressure pad incorporating the molecular sieve particles encapsulated in a mesh container for pressure application to a bleeding wound.

FIG. 4 is a perspective view of a bandage incorporating the molecular sieve particles in a mesh container for application to a bleeding wound.

### DETAILED DESCRIPTION OF THE PREFERRED EMBODIMENTS

Disclosed herein are hemostatic devices and hemostatic agents that are applicable to bleeding wounds to promote hemostasis. The hemostatic agents generally include quantities of particles having hemostatic qualities, such particles being contained within mesh bags, perforated containers, or similar devices that, when brought into contact with a bleeding wound, can minimize or stop blood flow by absorbing at least portions of the liquid phases of the blood, thereby facilitating clotting. Each particle includes a mixture of both a molecular sieve material component and a binder component. The particles are not limited to two-component mixtures, however, as other materials (e.g., anti-infective agents and the like) may be included as third or subsequent components.

In one preferred embodiment, the molecular sieve material is a zeolite and the binder component is a material having hemostatic properties. The devices and agents disclosed herein are not limited to zeolites, however, as other molecular sieve materials are within the scope of the present invention.

As used herein, the term “zeolite” refers to a crystalline form of aluminosilicate having one or more ionic species such as, for example, calcium and sodium moieties and the ability to be dehydrated without experiencing significant changes in the crystalline structure. Typically, the zeolite is a friable material that includes oxides of calcium, sodium, aluminum, and silicon in addition to water. The calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings. One preferred zeolite is that designated as “5 A,” which indicates a crystal size of about 5 angstroms and having a cubic crystalline structure defining round or substantially round openings.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides.

Preferred binders are clays having suitable hemostatic properties. The devices and agents disclosed herein are not limited to clays, however, as other materials are within the scope of the present invention. For example, bioactive glasses, siliceous oxides, diatomaceous earth, and combinations thereof may also be used in place of (or in addition to) the clay.

As used herein, the term “clay” refers to a crystalline form of hydrated aluminum silicate. The crystals of clay are irregularly shaped and insoluble in water. The combination of some types of clay with water may produce a mass having some degree of plasticity. Depending upon the type of clay, the combination thereof with water may produce a colloidal gel having thixotropic properties.

The clay utilized in the hemostatic agents and devices of the present invention is preferably kaolin, which is an aluminum phyllosilicate comprising about 50% alumina, about 50% silica, and trace impurities. The clay may be Edgar's plastic kaolin (hereinafter “EPK”), which is a water-washed kaolin clay that is mined and processed in and near Edgar, Fla. Edgar's plastic kaolin has desirable plasticity characteristics, is castable, and when mixed with water produces a thixotropic slurry. Other clays such as attapulgite and bentonite are also within the scope of the present invention and can be used individually, in combination with each other, or in combination with kaolin.

The EPK used in the present invention is particlized, dried, and powdered. In order to achieve a suitably homogenous mixture of the EPK for subsequent conversion into powder, a relatively high shear is applied to a mass of the EPK using a suitable mixing apparatus. Prior to shearing, the water content of the clay is measured and adjusted to be about 20% by weight to give a sufficiently workable mixture for extrusion and subsequent handling. The EPK is then worked into cakes and dried in ovens. Upon drying to a suitable moisture content, the cakes are then crushed into powder.

The zeolite/clay particles can be produced by any of several various methods. Such methods include mixing, extrusion, spheronizing, and the like. Equipment that can be utilized for the mixing, extruding, or spheronizing of the clay is available from Caleva Process Solutions Ltd. in Dorset, United Kingdom. Other methods include the use of a fluid bed or a pelletizing apparatus. Fluid beds for the production of particles are available from Glatt Air Technologies in Ramsey, N.J. Disk pelletizers for the production of clay particles are available from Feeco International, Inc., in Green Bay, Wis. Preferably, a mixture of the zeolite and the clay is extruded through a suitable pelletizing device. The present invention is not limited in this regard, however, as other devices and methods for producing particles of hemostatic agent are within the scope of the present invention.

As used herein, “particles” of hemostatic agent can include beads, pellets, granules, rods, or any other surface morphology or combination of surface morphologies. Irrespective of the surface morphology, the zeolite/clay particles are about 0.2 mm (millimeters) to about 10 mm, preferably about 0.5 mm to about 5 mm, and more preferably about 1 mm to about 2 mm in effective diameter.

In some embodiments of the present invention, the zeolite/clay particles may be fired to about 600 degrees C. to vitrify the clay portion. Vitrification is effected via repeated melting and cooling cycles to allow the EPK (or other clay material) to be converted into a glassy substance. With increasing numbers of cycles, the crystalline structure is broken down to result in an amorphous composition. The amorphous nature of the EPK allows it to maintain its structural integrity when subsequently wetted. As a result, the EPK resists the tendency to fall apart when wetted during use, for example, when applied to blood. In embodiments in which the zeolite is mixed with the EPK prior to vitrification, the zeolite is unaffected by the heating and cooling of the particle.

In other embodiments, the vitrification process may be foregone to provide friable particles that are soft and loosely packed. Packing each particle loosely with zeolite and clay allows the clay portion to crumble when applied to blood, thereby dispersing both the zeolite and the clay throughout the wound.

It is believed that the cellular clotting mechanisms of both zeolite and clay activate certain contact factors when applied to blood. More specifically, it is believed that zeolite and kaolin (particularly EPK) are different but complementary. While each material exhibits hemostatic qualities on its own, it is likely that the differences in the molecular structures of each initiate different mechanisms by which water in blood is absorbed to facilitate clotting functions.

Irrespective of the clotting mechanisms of the zeolite and the clay, in formulating the hemostatic agent for use with a hemostatic device, the zeolite and the clay are blended and particlized to produce a uniform, homogenous mixture of exothermic and non-exothermic material having a biocompatible aspect. The amounts of zeolite and clay for the particles are selected to provide a particular exotherm when the particles are applied to a bleeding wound. Variation in the amounts of each component allows any heat generated from the application of the hemostatic agent to a bleeding wound to be modulated as desired.

The zeolite/clay particles of the hemostatic agent may be mixed with, incorporate, or otherwise used in conjunction with other materials having the ability to be dehydrated without significant changes in crystalline structure while imparting beneficial qualities to the hemostatic agent. Such materials include, but are not limited to, magnesium sulfate, sodium metaphosphate, calcium chloride, dextrin, combinations of the foregoing materials, and hydrates of the foregoing materials.

Various other materials may also be mixed with, associated with, or incorporated into the zeolite/clay mixture of the hemostatic agent to maintain an antiseptic environment at the wound site or to provide functions that are supplemental to the clotting functions of the zeolite and the clay. Exemplary materials that can be used include, but are not limited to, pharmaceutically-active compositions such as wound healing agents, antibiotics, antifungal agents, anti-infective agents, antimicrobial agents, anti-inflammatory agents, analgesics, antihistamines (e.g., cimetidine, chloropheniramine maleate, diphenhydramine hydrochloride, and promethazine hydrochloride), compounds containing silver ions and/or copper ions, and the like. Other materials that can be incorporated to provide additional hemostatic functions include ascorbic acid, tranexamic acid, rutin, and thrombin. Botanical agents having desirable effects on the wound site may also be added.

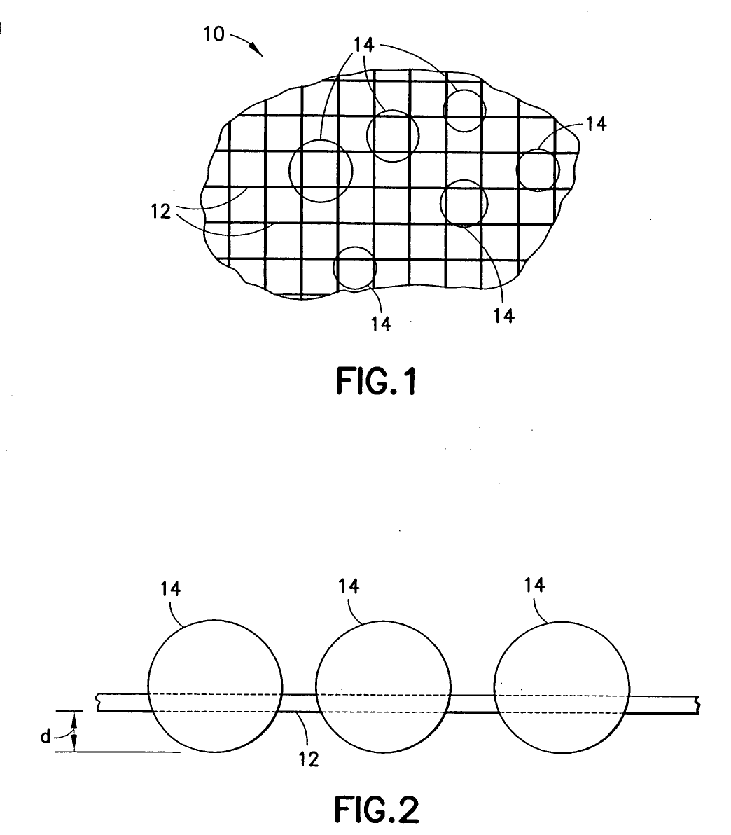

Referring now to FIG. 1, a hemostatic device into which the hemostatic agent is incorporated is shown. The device is a permeable pouch that allows liquid to enter to contact the hemostatic agent retained therein. Sealed packaging (not shown) provides a sterile environment for storing the hemostatic device until it can be used. The device, which is shown generally at 10 and is hereinafter referred to as “pouch 10,” comprises a screen or mesh 12 and the hemostatic agent 14 retained therein by the screen or mesh. The mesh 12 is closed on all sides and defines openings that are capable of retaining the hemostatic agent 14 therein while allowing liquid to flow through. As illustrated, the mesh 12 is shown as being flattened out, and only a few particles of hemostatic agent 14 are shown. The hemostatic agent 14 comprises the zeolite and clay (or other hemostatic material) particles as described herein.

The mesh 12 is defined by interconnected strands, filaments, or strips of material. The strands, filaments, or strips can be interconnected in any one or a combination of manners including, but not limited to, being woven into a gauze, intertwined, integrally-formed, and the like. Preferably, the interconnection is such that the mesh can flex while substantially maintaining the dimensions of the openings defined thereby. The material from which the strands, filaments or strips are fabricated may be a polymer (e.g., nylon, polyethylene, polypropylene, polyester, or the like), metal, fiberglass, or an organic substance (e.g., cotton, wool, silk, or the like).

Referring now to FIG. 2, the openings defined by the mesh 12 are dimensioned to retain the hemostatic agent 14 but to accommodate the flow of blood therethrough. Because the mesh 12 may be pulled tight around the hemostatic agent 14, the particles may extend through the openings by a distance d. If the particles extend through the openings, they are able to directly contact tissue to which the pouch 10 is applied. Thus, blood emanating from the tissue immediately contacts the hemostatic agent 14, and the water phase thereof is wicked into the zeolite and clay materials, thereby facilitating the clotting of the blood. However, it is not a requirement of the present invention that the particles protrude through the mesh.

To apply the pouch 10 to a bleeding wound, the pouch is removed from the packaging and placed on the bleeding wound. The hemostatic agent 14 in the mesh 12 contacts the tissue of the wound and/or the blood, and at least a portion of the liquid phase of the blood is adsorbed by the zeolite and clay of the particles, thereby promoting the clotting of the blood.

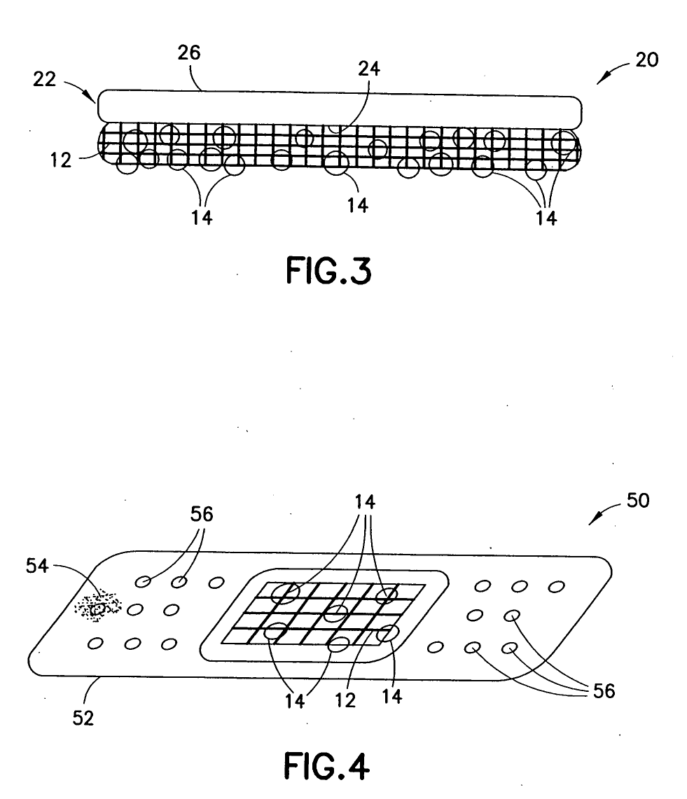

Another embodiment of the present invention is a pad which is shown at 20 with reference to FIG. 3 and is hereinafter referred to as “pad 20.” The pad 20 comprises the mesh 12, hemostatic agent 14 retained therein by the mesh 12, and a support 22 to which pressure may be applied in the application of the pad 20 to a bleeding wound. The mesh 12, as above, has openings that are capable of retaining the particles therein while allowing the flow of blood therethrough.

The mesh 12 is stitched, glued, clamped, or otherwise mounted to the support 22. The support 22 comprises an undersurface 24 against which the hemostatic agent 14 is held by the container 12 and a top surface 26. The undersurface 24 is impermeable to the hemostatic agent 14 (migration of the particles into the support 22 is prevented) and is further resistant to the absorption of water or other fluids. The top surface 26 is capable of having a pressure exerted thereon by a person applying the pad 20 to a bleeding wound or by a weight supported on the top surface 26. The entire support 22 is rigid or semi-rigid so as to allow the application of pressure while minimizing discomfort to the patient.

To apply the pad 20 to a bleeding wound, the pad 20 is removed from its packaging and placed on the bleeding wound. As with the pouch of the embodiment of FIGS. 1 and 2, the hemostatic agent 14 is either in direct contact with the tissue of the wound or is in direct contact with the blood. Pressure may be applied to the wound by pressing on the top surface 26 with a hand or by placing a weight on the surface, thereby facilitating the contact between the particles and the wound and promoting the adsorption of the liquid phase of the blood. The pad 20 (with or without a weight) may also be held onto the wound using a strapping device such as a belt, an elastic device, book-and-loop material, combinations of the foregoing devices and materials, and the like.

Referring now to FIG. 4, another embodiment of the present invention is a bandage, shown at 50, which comprises particles of the hemostatic agent 14 retained in a mesh 12 and mounted to a flexible substrate 52 that can be applied to a wound (for example, using a pressure-sensitive adhesive to adhere the bandage 50 to the skin of a wearer). The mesh 12 is stitched, glued, or otherwise mounted to a substrate 52 to form the bandage 50.

The substrate 52 is a plastic or a cloth member that is conducive to being retained on the skin of an injured person or animal on or proximate a bleeding wound. An adhesive 54 is disposed on a surface of the substrate 52 that engages the skin of the injured person or animal. Particularly if the substrate 52 is a non-breathable plastic material, the substrate may include holes 56 to allow for the dissipation of moisture evaporating from the skin surface.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A hemostatic agent, comprising:
a particle comprising,
a first component having hemostatic properties, and
a second component having hemostatic properties, said second component binding said first component;
wherein said first component and said second component are uniformly and homogenously mixed in amounts to modulate an exothermic reaction when said hemostatic agent is applied to blood; and
wherein when using said hemostatic agent to treat a bleeding wound, contacting said bleeding wound with said hemostatic agent causes blood flowing from said bleeding wound to clot.

2. The hemostatic agent of claim 1, wherein said first component is a zeolite.

3. The hemostatic agent of claim 1, wherein said second component is a clay.

4. The hemostatic agent of claim 3, wherein said clay is kaolin.

5. The hemostatic agent of claim 4, wherein said kaolin is Edgar's plastic kaolin.

6. The hemostatic agent of claim 3, wherein said clay is selected from the group consisting of attapulgite, bentonite, kaolin, and combinations of the foregoing.

7. The hemostatic agent of claim 1, wherein said particle is in a form of a bead, a rod, a granule, or has an irregular surface morphology.

8. The hemostatic agent of claim 1, wherein particle is about 0.2 mm to about 10 mm in effective diameter.

9. The hemostatic agent of claim 1, wherein particle is about 0.5 mm to about 5 mm in effective diameter.

10. The hemostatic agent of claim 1, wherein particle is about 1 mm to about 2 mm in effective diameter.

11. The hemostatic agent of claim 1, further comprising a third component incorporated into said particle, said third component having a property that is at least one of antibiotic, antifungal, anti-infective, antimicrobial, anti-inflammatory, analgesic, antihistamine, wound healing, and hemostatic.

12. The hemostatic agent of claim 1, wherein at least one of said first component and said second component is selected from the group consisting of bioactive glasses, siliceous oxides, diatomaceous earth, and combinations of the foregoing.

13. A device for promoting the clotting of blood, comprising:
a receptacle, at least a portion of said receptacle being defined by a mesh having openings therein,
a hemostatic agent retained in said receptacle, said hemostatic agent comprising particles, each particle having a first component having hemostatic properties and a second component having hemostatic properties, said second component binding said first component;
wherein said first component and said second component are uniformly and homogenously mixed in amounts to modulate an exothermic reaction when said hemostatic agent is applied to blood; and
wherein when treating a bleeding wound, application of said device causes at least a portion of said hemostatic agent to come into contact with blood through said openings.

14. The device for promoting the clotting of blood of claim 13, wherein said first component is a zeolite.

15. The device for promoting the clotting of blood of claim 13, wherein said second component is a clay.

16. The device for promoting the clotting of blood of claim 15, wherein said clay is selected from the group consisting of attapulgite, bentonite, kaolin, and combinations of the foregoing.

17. The device for promoting the clotting of blood of claim 15, wherein said clay is kaolin.

18. The device for promoting the clotting of blood of claim 13, wherein said particles are in the forms of beads, rods, granules, or have irregular surface morphologies.

19. The device for promoting the clotting of blood of claim 13, wherein at least one of said first component and said second component is selected from the group consisting of bioactive glasses, siliceous oxides, diatomaceous earth, and combinations of the foregoing.

20. The device for promoting the clotting of blood of claim 13, wherein said particle of said hemostatic agent further comprises a third component, said third component having a property that is at least one of antibiotic, antifungal, anti-infective, antimicrobial, anti-inflammatory, analgesic, antihistamine, wound healing, and hemostatic.

21. The device for promoting the clotting of blood of claim 13, wherein said mesh structure is flexible.

22. The device for promoting the clotting of blood of claim 13, wherein at least one particle of said hemostatic agent protrudes through one of said openings.

23. The device for promoting the clotting of blood of claim 13, wherein the effective diameters of the particles are about 0.2 mm to about 10 mm.

24. The device for promoting the clotting of blood of claim 13, wherein the effective diameters of the particles are about 0.5 mm to about 5 mm.

25. The device for promoting the clotting of blood of claim 13, wherein the effective diameters of the particles are about 1 mm to about 2 mm.

26. A pad for controlling bleeding, comprising:
a mesh structure defined by openings sized to accommodate the flow of blood therethrough;
a hemostatic agent retained in said mesh structure, said hemostatic agent comprising particles, each particle having a first component having hemostatic properties and a second component having hemostatic properties, said second component binding said first component;
wherein said first component and said second component are uniformly and homogenously mixed in amounts to modulate an exothermic reaction when said hemostatic agent is applied to blood; and
wherein when treating a bleeding wound, application of said pad causes at least a portion of said hemostatic agent to come into contact with blood through said openings.

27. The pad of claim 26, wherein said first component is a zeolite and wherein said second component is a clay.

28. The pad of claim 27, wherein said clay is selected from the group consisting of attapulgite, bentonite, kaolin, and combinations of the foregoing.

29. The pad of claim 27, wherein said clay is kaolin.

30. The pad of claim 26, wherein said particles are in the forms of beads, rods, granules, or have irregular surface morphologies.

31. The pad of claim 26, wherein at least one of said first component and said second component is selected from the group consisting of bioactive glasses, siliceous oxides, diatomaceous earth, and combinations of the foregoing.

32. The pad of claim 26, wherein said particle of said hemostatic agent further comprises a third component, said third component having a property that is at least one of antibiotic, antifungal, anti-infective, antimicrobial, anti-inflammatory, analgesic, antihistamine, wound healing, and hemostatic.

33. The pad of claim 26, wherein said particles of said hemostatic agent each have effective diameters of about 0.2 mm to about 10 mm.

34. The pad of claim 26, wherein said particles of said hemostatic agent each have effective diameters of about 0.5 mm to about 5 mm.

35. The pad of claim 26, wherein said particles of said hemostatic agent each have effective diameters of about 1 mm to about 2 mm.

36. The pad of claim 26, further comprising a support attached to said mesh structure, said support being configured to have a pressure applied thereto to enable said pad to be retained on a bleeding wound.

37. A bandage applicable to a bleeding wound, said bandage comprising:
a substrate;
a mesh mounted on said substrate, said mesh being defined by a plurality of members arranged to define openings, said openings being dimensioned to accommodate the flow of blood therethrough; and
a hemostatic agent retained in said mesh, said hemostatic agent comprising particles, each particle having a first component having hemostatic properties and a second component having hemostatic properties, said second component binding said first component, said first component and said second component being uniformly and homogenously mixed in amounts to modulate an exothermic reaction when said hemostatic agent is applied to blood.

38. The bandage of claim 37, further comprising an adhesive on said substrate, said adhesive being configured to facilitate the retaining of said bandage on the skin of a wearer.

39. The bandage of claim 37, wherein said first component is a zeolite and said second component is a clay.

40. The bandage of claim 39, wherein said clay is selected from the group consisting of attapulgite, bentonite, kaolin, and combinations of the foregoing.

41. The bandage of claim 39, wherein said clay is kaolin.

42. The bandage of claim 37, wherein said particles are in the forms of beads, rods, granules, or have irregular surface morphologies.

43. The bandage of claim 37, wherein at least one of said first component and said second component is selected from the group consisting of bioactive glasses, siliceous oxides, diatomaceous earth, and combinations of the foregoing.

44. The bandage of claim 37, wherein said particle of said hemostatic agent further comprises a third component, said third component having a property that is at least one of antibiotic, antifungal, anti-infective, antimicrobial, anti-inflammatory, analgesic, antihistamine, wound healing, and hemostatic.
